(19) United States
(12) Patent Application Publication
(10) Pub. No.: US 2018/0200302 A1
(43) Pub. Date: Jul. 19, 2018

(54) BONE MARROW STROMAL CELL DERIVED EXTRACELLULAR MATRIX PROTEIN EXTRACT AND USES THEREOF

(71) Applicant: STEMBIOSYS, INC., San Antonio, TX (US)

(72) Inventors: Rogelio ZAMILPA, San Antonio, TX (US); Edward S. Griffey, San Antonio, TX (US)

(73) Assignee: STEMBIOSYS, INC., San Antonio, TX (US)

(21) Appl. No.: 15/919,499

(22) Filed: Mar. 13, 2018

Related U.S. Application Data
(62) Division of application No. 15/472,484, filed on Mar. 29, 2017.
(60) Provisional application No. 62/315,460, filed on Mar. 30, 2016.

(51) Int. Cl. A61K 35/28; C12N 5/0775; A61L 27/12; A61L 27/36; C07K 14/47; A61K 47/02; A61K 35/12

(52) U.S. Cl. CPC A61K 35/28; C12N 5/0663; A61L 27/12; A61L 27/3608; A61L 27/3633; C07K 14/47; A61L 2430/02; A61L 27/3683; A61K 2035/124; C12N 2502/1394; C12N 2533/52; C12N 2533/90; A61K 47/02

(57) ABSTRACT

Disclosed are bone marrow stromal cell derived extracellular matrix protein extracts that are useful for the expansion and proliferation of mesenchymal stem cells and for various therapeutic applications.

### CROSS-REFERENCE TO RELATED APPLICATIONS

This application is a divisional of U.S. application Ser. No. 15/472,484, filed Mar. 29, 2017, which claims the benefit of U.S. Provisional Application No. 62/315,460 filed Mar. 30, 2016, the contents of which applications are incorporated herein in their entirety.

### BACKGROUND OF THE INVENTION

A. Field of the Invention

The invention generally relates to cell derived extracellular matrices and uses thereof.

B. Description of Relevant Art

Mesenchymal stem cells (MSC) have been shown be useful in a variety of therapeutic applications including promotion of tissue repair and regeneration, due to their ability to immunomodulate the microenvironment, stimulate angiogenesis, and give rise to multiple cell types. However, one of the obstacles associated with MSC therapies is obtaining relevant numbers of MSCs for clinical treatments. It has been found that extracellular matrices (ECM) generated by bone marrow stromal cells promote proliferation and maintain MSCs in their undifferentiated state as disclosed in U.S. Pat. No. 8,084,023, U.S. Pat. No. 8,388,947, and U.S. Pat. No. 8,961,955 all of which are herein incorporated by reference in their entirety. These particular ECMs are grown on and attached to substrates such as culture dishes. Other substrates to which the ECMs are attached include microcarriers, as disclosed in PCT application PCT/US2015/058335 herein incorporated by reference in its entirety. Notably, however, because of the fixed nature of this configuration, i.e. ECMs are physically attached to the substrates on which they are grown, use of these ECMs are currently limited for other applications such as adding the ECM directly into a cell growth medium as a supplement; mixing the ECM with other biologic materials or medical devices (i.e., a bone substitute material with delivery directly to the site of a bone injury); coating surfaces with the ECM that may not be conducive to attachment of the ECM producing cells; and incorporating the ECM into carriers such as gels, liquids, or powders, e.g. ceramic powders.

### SUMMARY OF THE INVENTION

The present invention provides a solution to the aforementioned limitations and deficiencies in the art relating to use of bone marrow stromal cell derived ECMs that are physically attached to substrates on which they were grown, i.e., the ECMs are in direct contact with the substrate. The solution is premised on the physical removal of the bone marrow stromal cell derived ECM from the substrate on which it was grown and removal of all or a portion of its soluble proteins, resulting in a bone marrow stromal cell derived ECM protein extract. For purposes of this invention, the aforementioned bone marrow stromal cell ECM (insoluble) protein extract is referred to and identified by any of the following terms: “bone marrow stromal cell derived extracellular matrix protein extract”, “bone marrow stromal cell derived ECM protein extract”, “marrow stromal cell derived extracellular matrix protein extract”, “marrow stromal cell derived ECM protein extract”, “extracellular matrix protein extract”, ECM (insoluble) protein extract, or “ECM protein extract”.

The bone marrow stromal cell derived ECM protein extract of the invention is an acellular three-dimensional (3D) matrix generated by bone marrow stromal cells that is no longer attached to the substrate on which it was grown, and where all or a portion of the soluble proteins originally present in the ECM have been removed. It was surprisingly discovered that the bone marrow stromal cell derived ECM protein extract of the invention demonstrated greater MSC stimulation than did the bone marrow stromal cell derived ECM protein with its soluble proteins still present. Also, the bone marrow stromal cell derived ECM protein extract of the invention demonstrated greater MSC stimulation than did the bone marrow stromal cell derived ECM still attached to the substrate on which it was grown. Without being bound to theory, the soluble proteins of the bone marrow stromal cell derived ECM may have an inhibitory effect on cell growth.

Use of the ECM protein extract of the invention has many advantages over use of an ECM still attached to the substrate on which it was grown. Non-limiting examples include: adding the ECM protein extract directly into a cell growth medium as a supplement; mixing the ECM protein extract with other biologic materials or medical devices (i.e., a bone substitute material with delivery directly to the site of a bone injury); dip coating various surfaces with the ECM protein extract rather than growing an ECM on a surface; and/or coating surfaces with the ECM protein extract that may not be conducive to attachment of the ECM producing cells. Additionally, the ECM protein extract can be incorporated into carriers such as gels, liquids, or powders, e.g. ceramic powders, for delivery of the ECM protein extract.

An important therapeutic use of the bone marrow stromal cell derived ECM protein extract is its use in bone tissue engineering such as bone formation, regeneration, and bonding. The bone marrow stromal cell derived ECM protein extract of the invention promoted greater bone regeneration in-vivo when combined with ceramic powders than other bone regeneration materials such as ceramic powders alone.

The bone marrow stromal cell derived ECM protein extract of the invention can also be used for proliferating and expanding MSCs in culture and maintaining the MSCs in an undifferentiated state in culture.

In one aspect of the invention, there is disclosed an extracellular matrix (ECM) protein extract comprising: a bone marrow stromal cell derived ECM grown on a substrate and comprising insoluble and soluble proteins, wherein the ECM is not attached to the substrate on which it was grown, and wherein all or a portion of the soluble proteins originally present in the ECM have been removed.

In another aspect of the invention, there is disclosed an extracellular matrix (ECM) protein extract consisting essentially of: a bone marrow stromal cell derived ECM grown on a substrate and comprising insoluble and soluble proteins, wherein the ECM is not attached to the substrate on which it was grown, and wherein all or a portion of the soluble proteins originally present in the ECM have been removed.

In another aspect of the invention, there is disclosed an extracellular matrix (ECM) protein extract consisting of: a bone marrow stromal cell derived ECM grown on a substrate and comprising insoluble and soluble proteins, wherein the ECM is not attached to the substrate on which it was grown, and wherein all or a portion of the soluble proteins originally present in the ECM have been removed.

In another aspect of the invention, there is disclosed a composition comprising an extracellular matrix (ECM) protein extract comprising, consisting essentially of, or consisting of: a bone marrow stromal cell derived ECM grown on a substrate and comprising insoluble and soluble proteins, wherein the ECM is not attached to the substrate on which it was grown, and wherein all or a portion of the soluble proteins originally present in the ECM have been removed. In some embodiments, the composition further comprises a carrier. In other embodiments, the carrier is a gel, aqueous liquid, or ceramic powder.

In another aspect of the invention, there is disclosed a bone marrow stromal cell derived extracellular matrix (ECM) protein extract made by the method comprising:
(a) obtaining viable bone marrow stromal cells,
(b) culturing the bone marrow stromal cells on a substrate to produce a 3D ECM on the substrate,
(c) decellularizing the bone marrow stromal cells from the ECM,
(d) physically removing the ECM from the substrate,
(e) contacting the ECM with an aqueous component with agitation to dissolve and disassociate the soluble proteins of the ECM, and
(f) removing the aqueous component from the remaining insoluble portion (protein extract) of the ECM.

In another aspect of the invention, there is disclosed a method of making a bone marrow stromal cell derived ECM protein extract, the method comprising:
(a) obtaining viable bone marrow stromal cells,
(b) culturing the bone marrow stromal cells on a substrate to produce a 3D ECM on the substrate,
(c) decellularizing the bone marrow stromal cells from the ECM,
(d) physically removing the ECM from the substrate,
(e) contacting the ECM with an aqueous component with agitation to dissolve and disassociate the soluble proteins of the ECM, and
(f) removing the aqueous component from the remaining insoluble portion (protein extract) of the ECM.

Steps (d) and (e) above can be performed concurrently. The physical removal of the ECM from the substrate in Step (d) does not include enzymatic digestion of the ECM. However, the physical removal of the ECM from the substrate in Step (d) does include mechanical removal of the ECM from the substrate, such as with a spatula or scraper; and/or removal of the ECM from the substrate with agitation, such as with a mixer, homogenizer or sonicator. The agitation in Step (e) can include mixing or homogenization which can be performed using sonication or physical mixing such as with a spatula or homogenizer, or other mixing/homogenization techniques known in the art.

As noted throughout the specification and claims, the ECM protein extracts of the present invention comprise a bone marrow stromal cell derived ECM wherein the ECM is not attached to the substrate on which it was grown. The phrase “wherein the ECM is not attached to the substrate on which it was grown” refers, for example, to the substrate used in the above process steps (b). In certain non-limiting embodiments of the present invention, the produced ECM protein extracts of the present invention can be further processed such that they are subsequently attached to another substrate (e.g., a substrate that is different than the substrate on which the extract was grown). The additional substrate can be the same type of substrate on which the extract was grown.

All or a portion of the soluble proteins originally present in the bone marrow stromal cell derived ECM are removed from the ECM resulting in the ECM protein extract of the invention. When the bone marrow stromal cell derived ECM is physically removed from the substrate on which it was grown and contacted with an aqueous component with agitation, the ECM is broken into pieces and some of the proteins will unravel and become dissociated or dissolved from the ECM and remain in the aqueous component. The agitation breaks up the ECM into pieces and also allows greater surface contact of the ECM with the aqueous component than would be with simply washing the surface of the ECM while still attached to the substrate. This aqueous component/soluble protein mixture is removed from the insoluble portion of the ECM. The insoluble portion is the ECM protein extract. Thus, the terms “soluble protein” or “soluble proteins” when used in the context of this invention means water-soluble proteins as well as light proteins and protein fragments suspended in and/or dissolved in the aqueous component. It is contemplated that the removed aqueous component/soluble proteins mixture can have research, clinical, and therapeutic applications.

In another aspect of the invention, there is disclosed a method for expanding mesenchymal stem cells (MSCs), the method comprising culturing the MSCs with a composition comprising an extracellular matrix (ECM) protein extract comprising, consisting essentially of, or consisting of: a bone marrow stromal cell derived ECM grown on a substrate and comprising insoluble and soluble proteins, wherein the ECM is not attached to the substrate on which it was grown, and wherein all or a portion of the soluble proteins originally present in the ECM have been removed.

In another aspect of the invention, there is disclosed a bone forming composition comprising an extracellular matrix (ECM) protein extract comprising, consisting essentially of, or consisting of: a bone marrow stromal cell derived ECM grown on a substrate and comprising insoluble and soluble proteins, wherein the ECM is not attached to the substrate on which it was grown, and wherein all or a portion of the soluble proteins originally present in the ECM have been removed. In some embodiments, the composition further comprises a carrier. In other embodiments, the carrier is a gel, aqueous liquid, or ceramic powder. In still other embodiments, the ceramic powder is hydroxyapatite or hydroxyapatite/tricalcium phosphate.

In another aspect of the invention, there is disclosed a method of generating bone in a subject comprising administering to a subject a composition comprising an extracellular matrix (ECM) protein extract comprising, consisting essentially of, or consisting of: a bone marrow stromal cell derived ECM grown on a substrate and comprising insoluble and soluble proteins, wherein the ECM is not attached to the substrate on which it was grown, and wherein all or a portion of the soluble proteins originally present in the ECM have been removed. In some embodiments, the composition further comprises a carrier. In other embodiments, the carrier is a gel, aqueous liquid, or ceramic powder. In still other embodiments, the ceramic powder is hydroxyapatite or hydroxyapatite/tricalcium phosphate.

In some embodiments, the bone marrow stromal cells used to make the ECM protein extract are murine, rabbit, cat, dog, pig, equine, or primate. In other embodiments, the bone marrow stromal cells are human. In other embodiments, the bone marrow stromal cells are equine. In other embodiments, the bone marrow stromal cells are murine. In still other embodiments, the bone marrow stromal cells are isolated bone marrow mesenchymal stem cells.

In some embodiments, the bone marrow stromal cell derived ECM protein extract is produced under normoxic conditions.

Also disclosed are the following Embodiments 1-18 of the present invention. Embodiment 1 is an extracellular matrix (ECM) protein extract comprising a bone marrow stromal cell derived ECM grown on a substrate and comprising insoluble and soluble proteins, wherein the ECM is not attached to the substrate on which it was grown, and wherein all or a portion of the soluble proteins originally present in the ECM have been removed. Embodiment 2 is a composition comprising the extracellular matrix (ECM) protein extract of Embodiment 1. Embodiment 3 is the composition of Embodiment 2, wherein the composition further comprises a carrier. Embodiment 4 is the composition of Embodiment 3, wherein the carrier is a gel, aqueous liquid, or ceramic powder. Embodiment 5 is an extracellular matrix (ECM) protein extract made by the method comprising: (a) obtaining viable bone marrow stromal cells; (b) culturing the bone marrow stromal cells on a substrate to produce a 3D ECM on the substrate; (c) decellularizing the bone marrow stromal cells from the ECM; (d) physically removing the ECM from the substrate; (e) contacting the ECM with an aqueous component with agitation to dissolve and disassociate the soluble proteins of the ECM; and (f) removing the aqueous component from the remaining insoluble portion (protein extract) of the ECM. Embodiment 6 is the ECM protein extract of Embodiments 1 or 5, wherein the substrate is a cell culture container, a plastic cover slip, or microcarriers. Embodiment 7 is the ECM protein extract of any one of Embodiments 1, 5, or 6, wherein the substrate is pre-coated with fibronectin. Embodiment 8 is a method of making an extracellular matrix (ECM) protein extract, the method comprising: (a) obtaining viable bone marrow stromal cells; (b) culturing the bone marrow stromal cells on a substrate to produce a 3D ECM on the substrate; (c) decellularizing the bone marrow stromal cells from the ECM; (d) physically removing the ECM from the substrate; (e) contacting the ECM with an aqueous component with agitation to dissolve and disassociate the soluble proteins of the ECM; and (f) removing the aqueous component from the remaining insoluble portion (protein extract) of the ECM. Embodiment 9 is the method of Embodiment 8, wherein the substrate is a cell culture container, a plastic cover slip, or microcarriers. Embodiment 10 is the method of Embodiments 8 or 9, wherein the substrate is pre-coated with fibronectin. Embodiment 11 is a method for expanding mesenchymal stem cells (MSCs), the method comprising culturing the MSCs with the composition of Embodiment 2. Embodiment 12 is a bone forming composition comprising the ECM protein extract of Embodiment 1. Embodiment 13 is the composition of Embodiment 12, wherein the composition further comprises a carrier. Embodiment 14 is the composition of Embodiment 13, wherein the carrier is a gel, aqueous liquid, or ceramic powder. Embodiment 15 is the composition of Embodiment 14, wherein the ceramic powder is hydroxyapatite or hydroxyapatite/tricalcium phosphate. Embodiment 16 is a method of generating bone in a subject comprising administering to a subject the composition of any of Embodiments 12 through 15. Embodiment 17 is the ECM protein extract of Embodiments 1 or 5, wherein the ECM protein extract comprises one or more of Alpha-1-antiproteinase, Alpha-2-HS-glycoprotein, Alpha-2-HS-glycoprotein precursor, Alpha-2-macroglobulin, Alpha-actinin-1, Annexin A2, Biglycan, Caveolin-1, Collagen alpha-1(I), Collagen alpha-1(II), Collagen alpha-1(III), Collagen alpha-1(VI), Collagen alpha-1(XII), Collagen alpha-1(XIV), Collagen alpha-2(I), Collagen alpha-2(V), Collagen alpha-2(VI), Collagen alpha-3(VI), Collagen type I, Collagen type III, Collagen type IV, Collagen type V, Collagen type VI, Decorin, Elongation factor 1-alpha, EMILIN-1, Endoplasmin, Fibrinogen, Fibronectin, Fibulin-1, Fibulin-2, Galectin-1-Homo sapiens (Human), Interferon-induced GTP-binding, Lamin-A/C, Laminin, LIM domain and actin-binding protein 1, Pentraxin-related, Periostin, Periostin precursor (PN), Perlecan, Plasminogen, Plectin, Profilin-1, Rubber elongation factor protein, Serine protease, Serpin H1, Serum albumin, Syndecan-1, Tenascin precursor (TN) (Human), Thrombospondin-1, Transforming growth factor-beta-induced protein, Transgelin, Vimentin. Embodiment 18 is the ECM protein extract of Embodiments 1 or 5, wherein the all or portion of the removed soluble proteins originally present in the ECM comprise one or more of Alpha-1-antiproteinase, Alpha-2-HS-glycoprotein, Alpha-2-HS-glycoprotein precursor, Alpha-2-macroglobulin, Alpha-actinin-1, Annexin A2, Biglycan, Caveolin-1, Collagen alpha-1(I), Collagen alpha-1(II), Collagen alpha-1(111), Collagen alpha-1(VI), Collagen alpha-1(XII), Collagen alpha-1(XIV), Collagen alpha-2(I), Collagen alpha-2(V), Collagen alpha-2(VI), Collagen alpha-3(VI), Collagen type I, Collagen type III, Collagen type IV, Collagen type V, Collagen type VI, Decorin, Elongation factor 1-alpha, EMILIN-1, Endoplasmin, Fibrinogen, Fibronectin, Fibulin-1, Fibulin-2, Galectin-1-Homo sapiens (Human), Interferon-induced GTP-binding, Lamin-A/C, Laminin, LIM domain and actin-binding protein 1, Pentraxin-related, Periostin, Periostin precursor (PN), Perlecan, Plasminogen, Plectin, Profilin-1, Rubber elongation factor protein, Serine protease, Serpin H1, Serum albumin, Syndecan-1, Tenascin precursor (TN) (Human), Thrombospondin-1, Transforming growth factor-beta-induced protein, Transgelin, Vimentin.

The term “mammal” or “mammalian” includes but is not limited to murine (e.g., rats, mice) mammals, rabbits, cats, dogs, pigs, equine (e.g., horses, donkeys) mammals, and primates (e.g., monkey, apes, humans). In particular aspects in the context of the present invention, the mammal can be a murine mammal, an equine mammal, or a human.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The use of the word “a” or “an” when used in conjunction with the terms “comprising”, “having”, “including”, or “containing” (or any variations of these words) may mean “one,” but it is also consistent with the meaning of “one or more,” “at least one,” and “one or more than one.”

The compositions and methods for their use can “comprise,” “consist essentially of,” or “consist of” any of the ingredients or steps disclosed throughout the specification.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Total cell number after MSC stimulation with ECM protein extract, ECM on substrate (positive control), ECM with soluble proteins, supernatant, and negative control (2-D culture dish).

FIG. 2: Absolute SSEA4 positive cell number after MSC stimulation with ECM protein extract, ECM on substrate (positive control), ECM with soluble proteins, supernatant, and negative control (2-D culture dish).

FIG. 3: Total cell number after MSC stimulation with varying concentrations of ECM protein extract, positive control, and negative control.

FIG. 4: Absolute SSEA4 positive cell number after MSC stimulation with varying concentrations of ECM protein extract, positive control, and negative control.

FIG. 5: Total cell number after MSC stimulation with varying concentrations of ECM protein extract, positive control, and negative control—matched lots of ECM.

FIG. 6: Absolute SSEA4 positive cell number after MSC stimulation with varying concentrations of ECM protein extract, positive control, and negative control—matched lots of ECM.

FIG. 7: X-ray images at 2-weeks for Group A (control).

FIG. 8: X-ray images at 4-weeks for Group A (control).

FIG. 9: X-ray images at 2-weeks for Group B (HA/TCP).

FIG. 10: X-ray images at 4-weeks for Group B (HA/TCP).

FIG. 11: X-ray images at 2-weeks for Group C (HA/TCP+ECM protein extract).

FIG. 12: X-ray images at 4-weeks for Group C (HA/TCP+ECM protein extract).

FIG. 13: Micro-CT images of Group A (control), Group B (HA/TCP) and Group C (HA/TCP+ECM protein extract) for the first 6 animals of the study after 4 weeks.

FIG. 14: Micro-CT composite images of Group A (control), Group B (HA/TCP) and Group C (HA/TCP+ECM protein extract) for the first 6 animals of the study after 4 weeks.

FIG. 15: Bone volume in ROI for the first 6 animals of the study after 4 weeks.

### DETAILED DESCRIPTION OF THE INVENTION

### A. Bone Marrow Stromal Cell Derived Extracellular Matrix (ECM) Protein Extract

The bone marrow stromal cell derived ECM protein extract of the invention is a three-dimensional (3D) ECM generated by bone marrow stromal cells, where the ECM is not attached to the substrate on which it was grown, and where all or a portion of the soluble proteins originally present in the ECM have been removed. Thus, the bone marrow stromal cell derived ECM protein extract has a different make-up from the original bone marrow stromal cell derived ECM.

The cells used to produce the ECM protein extract are stromal cells obtained from mammalian bone marrow. Marrow stromal cells can be obtained from various sources, such as, for example, iliac crest, femora, tibiae, spine, rib, or other medullary spaces. Marrow stromal cells can be obtained and cultured by common methods that are apparent to one of skill in the relevant art. The bone marrow stromal cells contain MSCs and other cells such as fibroblasts, adipocytes, macrophages, osteoblasts, osteoclasts, endothelial stem cells, and endothelial cells. The MSCs present in bone marrow can be isolated from the other cells present in bone marrow, and the isolated MSCs can be used as the bone marrow stromal cells to form the bone marrow stromal cell derived ECM protein extract. The bone marrow stromal cells can from various mammalian species. Non-limiting examples are human, primate, murine, equine, rabbit, cat, dog, or pig.

The bone marrow stromal cell derived ECM protein extract is comprised of various proteins. The components of the ECM protein extract can be identified by methods known in the art and can include immunohistochemical staining and mass spectroscopy. The bone marrow stromal cell derived ECM protein extract, can include, but is not limited to, the following components listed in Table 1.

TABLE 1

Alpha-1-antiproteinase
Alpha-2-HS-glycoprotein
Alpha-2-HS-glycoprotein precursor
Alpha-2-macroglobulin
Alpha-actinin-1
Annexin A2
Biglycan
Caveolin-1
Collagen alpha-1(I)
Collagen alpha-1(II)
Collagen alpha-1(III)
Collagen alpha-1(VI)
Collagen alpha-1(XII)
Collagen alpha-1(XIV)
Collagen alpha-2(I)
Collagen alpha-2(V)
Collagen alpha-2(VI)
Collagen alpha-3(VI)
Collagen type I
Collagen type III
Collagen type IV
Collagen type V
Collagen type VI
Decorin
Elongation factor 1-alpha
EMILIN-1
Endoplasmin
Fibrinogen
Fibronectin
Fibulin-1
Fibulin-2
Galectin-1 - Homo sapiens (Human)
Interferon-induced GTP-binding
Lamin-A/C
Laminin
LIM domain and actin-binding protein 1
Pentraxin-related
Periostin
Periostin precursor (PN)
Perlecan
Plasminogen
Plectin
Profilin-1
Rubber elongation factor protein
Serine protease
Serpin H1
Serum albumin
Syndecan-1
Tenascin precursor (TN) (Human)
Thrombospondin-1
Transforming growth factor-beta-induced protein
Transgelin
Vimentin

The bone marrow stromal cell derived ECM protein extract can include any combination of any components from Table 1.

The bone marrow stromal cell derived ECM protein extract can be produced by the following process:
(a) obtaining viable bone marrow stromal cells,
(b) culturing the bone marrow stromal cells on a substrate to produce a 3D ECM on the substrate,
(c) decellularizing the bone marrow stromal cells from the ECM,
(d) physically removing the ECM from the substrate,
(e) contacting the ECM with an aqueous component with agitation to dissolve and dissociate the soluble proteins of the ECM, and
(f) removing the aqueous component from the remaining insoluble portion (protein extract) of the ECM.

Regarding step (a), marrow stromal cells can be obtained from various sources, such as, for example, iliac crest, femora, tibiae, spine, rib, or other medullary spaces. Marrow stromal cells can be obtained and cultured by common methods that are apparent to one of skill in the relevant art. The bone marrow stromal cells contain MSCs and other cells such as fibroblasts, adipocytes, macrophages, osteoblasts, osteoclasts, endothelial stem cells, and endothelial cells. The MSCs present in bone marrow can be isolated from the other cells present in bone marrow, and the isolated MSCs can be used as the bone marrow stromal cells to form the bone marrow stromal cell derived ECM protein extract. The bone marrow stromal cells can from various mammalian species. Non-limiting examples are human, primate, murine, equine, rabbit, cat, dog, or pig.

Step (b) can be performed using the culture methods and techniques disclosed in U.S. Pat. No. 8,084,023, U.S. Pat. No. 8,388,947, and U.S. Pat. No. 8,961,955 all of which are herein incorporated by reference in their entirety; and other culture methods and techniques known in the art. An example of a method for producing the ECM of Step (b) followed by decellularizing the ECM in Step (c) is as follows: Freshly isolated murine femoral marrow cells are seeded onto tissue culture plastic at 3×10⁵ cells/cm², and cultured for seven days in α-MEM (Thermo-Fisher Scientific, Grand Island, N.Y.), supplemented with glutamine (2 mM), penicillin (100 U/ml), streptomycin (100 μg/ml) (Sigma Chemical Company, St. Louis, Mo.), and 15% pre-selected fetal bovine serum (FBS, Atlanta Biologicals, Lawrenceville, Ga.). Then the cells are seeded onto THERMANOX® plastic cover slips coated with fibronectin at 1×10⁴ cells/cm², and cultured for seven days in the supplemented α-MEM medium described above. Then ascorbic acid (50 μg/ml) (Sigma Chemical Company, St. Louis, Mo.) is added to the cell cultures for an additional eight days. After extensive washing with PBS, cells are removed from the ECM by incubation with 0.5% Triton X-100 containing 20 mM NH₄OH in PBS for five minutes at 37° C. The ECM is then treated with DNase at 100 μg/ml (Sigma Chemical Company, St. Louis, Mo.) for one hour at 37° C. The plates are washed with PBS three times, then 2.0 ml of PBS containing 50 μg/ml gentamicin and 0.25 μg/ml fungizone is added to the plates. The culturing of the marrow stromal cells can take place under normoxic conditions, i.e. 20-21% oxygen in the atmosphere, and can further include conditions at 37° C., 5% CO2, and 90% humidity. The substrate in Step (b) can be any substrate used in cell culture for the production of cell derived ECMs. Non-limiting examples of substrates include cell culture containers, e.g., tissue culture dishes and flasks, vats and reactors; plastic cover slips, e.g., THERMANOX Coverslips; Poly(Lactide-Co-Glycolide) substrates; synthetic hydrogels, e.g., polyacrylamide, PEG; collagenous scaffolds; and microcarriers, e.g., CYTODEX 1. The substrates may be pre-coated with proteins such as fibronectin prior to the culturing of the marrow stromal cells.

Steps (d) and (e) can be performed concurrently. The physical removal of the ECM from the substrate in Step (d) does not include enzymatic digestion of the ECM to remove it. However, the physical removal of the ECM from the substrate in Step (d) does include mechanical removal of the ECM from the substrate, such as with a spatula or scraper; and/or removal of the ECM from the substrate with agitation, such as with a mixer, homogenizer or sonicator. The agitation in Step (e) can include mixing or homogenization which can be performed using sonication or physical mixing such as with a spatula or homogenizer, or other mixing/homogenization techniques known in the art.

Step (f) can be performed using centrifugation or filtration, or other separation methods known in the art.

The process may further comprise irradiation after steps (b), (c), (d), (e), or (f).

The bone marrow stromal cell derived ECM protein extract can be sterile. It can be sterilized by irradiation; chemical sterilization, e.g., ethylene oxide; heat, e.g., autoclave; or other sterilization means. The bone marrow stromal cell derived ECM protein extract can be lyophilized.

Decellularizing the ECM of the bone marrow stromal cells can include removing the viable marrow stromal cells or rendering the marrow cells non-viable. The bone marrow stromal cells can be decellularized from the ECM by using methods known in the art and can include, but are not limited to lysing the marrow stromal cells and then removing the lysed marrow stromal cells by washing. Various substances can be used to remove the marrow stromal cells from the ECM and include TRITON X-100 and ammonium hydroxide in PBS buffer. After the ECM has been decellularized of marrow stromal cells, the resulting ECM is essentially free of marrow stromal cells.

The aqueous component can be water, an aqueous solution such as a buffer, or an aqueous-based culture medium. The aqueous component can be free of enzymes.

All or a portion of the soluble proteins originally present in the bone marrow stromal cell derived ECM are removed from the ECM resulting in the ECM protein extract of the invention. When the bone marrow stromal cell derived ECM is physically removed from the substrate on which it was grown and contacted with an aqueous component with agitation, the ECM is broken into pieces and some of the proteins will unravel and become dissociated or dissolved from the ECM and remain in the aqueous component. The agitation breaks up the ECM into pieces and also allows greater surface contact of the ECM with the aqueous component than would be with simply washing the surface of the ECM while still attached to the substrate. This aqueous component/soluble protein mixture is removed from the insoluble portion of the ECM. The insoluble portion is the ECM protein extract. Thus, the terms “soluble protein” or “soluble proteins” when used in the context of this invention means water-soluble proteins as well as light proteins and protein fragments suspended in and/or dissolved in the aqueous component. It is contemplated that the removed aqueous component/soluble proteins mixture can have research, clinical, and therapeutic applications. The soluble proteins present in the aqueous component/soluble protein mixture can include any combination of any components from Table 1.

The amount of the soluble proteins that are removed from the bone marrow stromal cell ECM can be all (100%); or a portion of the soluble proteins originally present in the ECM, i.e., from 95 to 100%, or from 90 to 100%, or from 85 to 100%, or from 80 to 100%, or from 75 to 100%, or from 70 to 100%, or from 65 to 100%, or from 60 to 100%, or from 55 to 100%, or from 50 to 100%, or from 45 to 100%, or from 40 to 100%, or from 35 to 100%, or from 30 to 100%, or from 25 to 100%, or from 20 to 100%, or from 15 to 100%, or from 10 to 100%, or from 5 to 100%, or from 1 to 100%, or from 85 to 90%, or from 80 to 90%, or from 75 to 90%, or from 70 to 90%, or from 65 to 90%, or from 60 to 90%, or from 55 to 90%, or from 50 to 90%, or from 45 to 90%, or from 40 to 90%, or from 35 to 90%, or from 30 to 90%, or from 25 to 90%, or from 20 to 90%, or from 15 to 90%, or from 10 to 90%, or from 5 to 90%, or from 1 to 90%, or from 75 to 80%, or from 70 to 80%, or from 65 to 80%, or from 60 to 80%, or from 55 to 80%, or from 50 to 80%, or from 45 to 80%, or from 40 to 80%, or from 35 to 80%, or from 30 to 80%, or from 25 to 80%, or from 20 to 80%, or from 15 to 80%, or from 10 to 80%, or from 5 to 80%, or from 1 to 80%, or from 65 to 70%, or from 60 to 70%, or from 55 to 70%, or from 50 to 70%, or from 45 to 70%, or from 40 to 70%, or from 35 to 70%, or from 30 to 70%, or from 25 to 70%, or from 20 to 70%, or from 15 to 70%, or from 10 to 70%, or from 5 to 70%, or from 1 to 70%, or from 55 to 60%, or from 50 to 60%, or from 45 to 60%, or from 40 to 60%, or from 35 to 60%, or from 30 to 60%, or from 25 to 60%, or from 20 to 60%, or from 15 to 60%, or from 10 to 60%, or from 5 to 60%, or from 1 to 60%, 45 to 50%, or from 40 to 50%, or from 35 to 50%, or from 30 to 50%, or from 25 to 50%, or from 20 to 50%, or from 15 to 50%, or from 10 to 50%, or from 5 to 50%, or from 1 to 50%, or from 35 to 40%, or from 30 to 40%, or from 25 to 40%, or from 20 to 40%, or from 15 to 40%, or from 10 to 40%, or from 5 to 40%, or from 1 to 40%, or from 25 to 30%, or from 20 to 30%, or from 15 to 30%, or from 10 to 30%, or from 5 to 30%, or from 1 to 30%, or from 20 to 25%, or from 15 to 25%, or from 10 to 25%, or from 5 to 25%, or from 1 to 25%, or from 15 to 20%, or from 10 to 20%, or from 5 to 20%, or from 1 to 20%, or from 10 to 15%, or from 5 to 15%, or from 1 to 15%, or from 5 to 10%, or from 1 to 10%, or from 1 to 5%.

Various commercially available cell culture media, e.g., α-MEM culture media (Thermo Fisher Scientific, Grand Island, N.Y.), can be used for culturing the bone marrow stromal cells and can also be the aqueous component for dissolving the water-soluble constituents of the ECM. The commercially available culture medium can be modified by adding various supplemental substances to the medium, e.g. sodium bicarbonate, L-glutamine, penicillin, streptomycin, Amphotericin B and/or serum. The serum can be fetal bovine serum. The medium can also be serum free. Additionally, substances such as L-ascorbic acid can be added to the medium or modified medium to induce cell production of an ECM.

### B. Methods to Expand and Proliferate Mammalian MSCs

Methods to expand and proliferate mammalian MSCs in an undifferentiated state include obtaining mammalian MSCs and culturing them with the bone marrow stromal cell derived ECM protein extract of the invention.

The culture of the mammalian MSCs can take place under normoxic conditions.

Mammalian MSCs can be obtained from various sources including, but not limited to bone marrow. Bone marrow may be obtained from various sources, such as, for example, iliac crest, femora, tibiae, spine, rib, or other medullary spaces. Mammalian MSCs can be obtained from other sources including, but are not limited to, embryonic yolk sac, placenta, umbilical cord tissues, umbilical cord blood, periosteum, trabecular bone, adipose tissue, synovium, skeletal muscle, deciduous teeth, fetal pancreas, lung, liver, amniotic fluid, and fetal and adolescent skin and blood. Methods for isolating and establishing cultures of MSCs are generally known to those of skill in the relevant art. Novel methods for isolating MSCs from umbilical cord blood are disclosed in US patent publication 2012/0142102, herein incorporated by reference in its entirety.

In some embodiments, the mammalian MSCs are human MSCs.

### C. Tissue Engineering

The bone marrow stromal cell derived ECM protein extract of the invention is useful in various tissue engineering applications such as bone and cartilage regeneration, and bone bonding. In-vivo studies have shown that the bone marrow stromal cell derived ECM protein extract when combined with hydroxyapatite/tricalcium phosphate (HA/TCP) showed greater bone volume regeneration as compared to HA/TCP alone.

The bone marrow stromal cell derived ECM protein extract can be combined with carriers and bone regeneration materials or used alone for bone tissue engineering applications. Non-limiting examples of carriers and bone regeneration materials include ceramic powders such as HA or HA/TCP; gels; and aqueous liquids. In some embodiments, the bone marrow stromal cell derived ECM protein extract is combined with HA or HA/TCP.

### EXAMPLES

The following examples are included to demonstrate certain non-limiting aspects of the invention. It should be appreciated by those of skill in the art that the techniques disclosed in the examples that follow represent techniques discovered by the applicants to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

### Production of a Bone Marrow Stromal Cell Derived ECM Protein Extract from Bone Marrow Stromal Cell Derived ECM

A bone marrow stromal cell derived ECM protein extract was made form a decellularized bone marrow stromal cell ECM using the following procedure:
(a) One ml of serum free medium (MEM alpha Medium 078-5077) was added to each of three 150 mm culture dishes where a bone marrow stromal cell derived ECM was previously produced and the viable stromal cells had been removed.
(b) The ECM of the 1^{st} culture dish was mechanically scrapped with a putty spatula to loosen the ECM from the surface of the dish and the contents were mixed with the spatula.
(c) The ECM/medium mixture of the 1^{st} dish was then decanted into the 2^{nd} culture dish.
(d) The ECM of the 2^{nd} dish was scrapped with the spatula and the contents of the 2^{nd} dish were mixed with the spatula.
(e) The mixture of the 2^{nd} dish was then decanted into the 3^{rd} culture dish.
(f) The ECM of the 3^{rd} culture dish was scrapped with the spatula and the contents of the 3^{rd} dish were mixed with the spatula.
(g) The mixture of the 3^{rd} dish was then transferred into a 15 ml conical tube.
(h) Each of the three 150 mm culture dishes was washed with 0.5 ml of serum free medium and the washings were added to the 15 ml conical tube containing the ECM/medium mixture.
(i) The 15 ml conical tube was sonicated 4 times for 2 minutes each time with a 1-minute break between times at 90% amplification with pulse 01, 01.
(j) A sample of the mixture from the 15 ml conical tube was pipetted into a 1.5 ml Eppendorf tube and centrifuged at 15,000×g for 5 minutes.
(k) The supernatant containing the soluble proteins was removed leaving the insoluble pellet which is the ECM protein extract.

### Example 2

### In-Vitro MSC Stimulation with Bone Marrow Stromal Cell Derived ECM Protein Extract

Bone marrow MSCs were seeded at 6000 cells/cm² in tissue culture dishes with the following culture medium iterations:
(a) 18 μg/ml of bone marrow stromal cell derived ECM protein extract (pellet from Example 1 k) suspended in culture medium
(b) 18 μg/ml of bone marrow stromal cell derived ECM where the soluble proteins were not removed (complete extract from Example 1i) suspended in culture medium
(c) 3 μg/ml of supernatant (from Example 1k) added to culture medium
(d) a negative control with culture medium alone (designated as 2D)
(e) a positive control with seeding on a bone marrow stromal cell derived ECM grown on and attached to the culture dish substrate (designated as HPME) with culture medium.

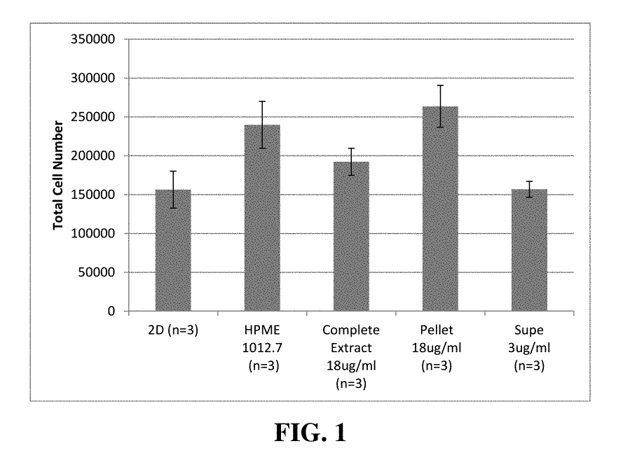

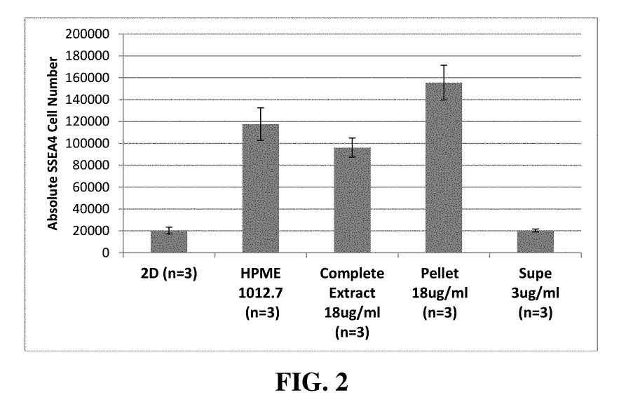

The dishes were incubated at 37° C. for 96 hours after which the cells were detached and counted. The cells were analyzed for SSEA4 expression (MSC marker) using flow cytometry. The data presented are total cell number in FIG. 1 and absolute SSEA4 positive cell number in FIG. 2.

As can be seen from FIG. 2, the greatest stimulation of the MSCs occurred with the bone marrow stromal cell derived ECM protein extract (iteration (a) designated as “Pellet”).

### Example 3

### In-Vitro MSC Stimulation by ECM Protein Extract Vs. Controls Study 1

Bone marrow MSCs were seeded at 6000 cells/cm² in tissue culture dishes with the following culture medium iterations:
(a) 10 μg/ml of bone marrow stromal cell derived ECM protein extract suspended in culture medium
(b) 20 μg/ml of bone marrow stromal cell derived ECM protein extract suspended in culture medium
(c) 40 μg/ml of bone marrow stromal cell derived ECM protein extract suspended in culture medium
(d) a negative control with culture medium alone (designated as 2D)
(e) a positive control with seeding on a bone marrow stromal cell derived ECM grown on and attached to the culture dish substrate (designated as 1012-4 HPME) with culture medium.
   Note: The bone marrow stromal cells used to produce the ECM protein extract and the ECM attached to the substrate in Study 1 were from the same donor; however, the ECM protein extract was not made from the same lot of ECM attached to the substrate.

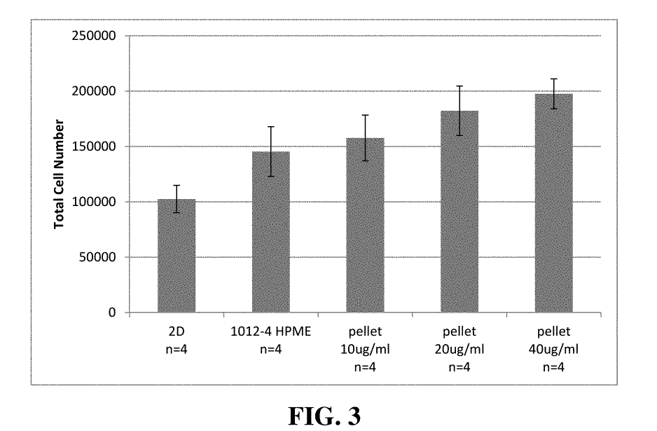

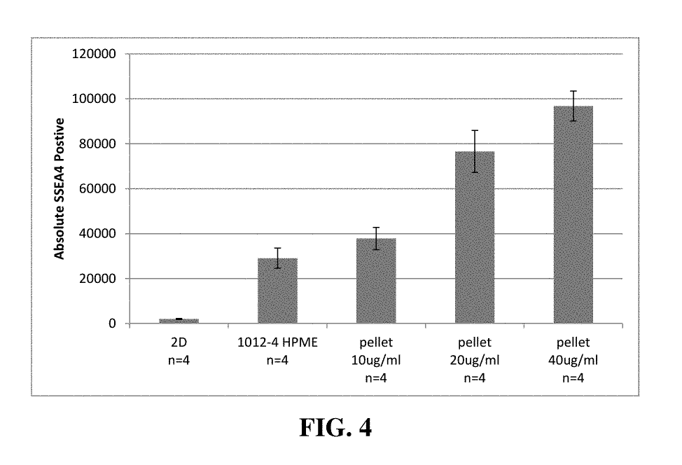

The dishes were incubated at 37° C. for 96 hours after which the cells were detached and counted. The cells were analyzed for SSEA4 expression (MSC marker) using flow cytometry. The data presented are total cell number in FIG. 3 and absolute SSEA4 positive cell number in FIG. 4.

As can be seen from FIG. 4, the bone marrow stromal cell derived ECM protein extract showed greater stimulation of MSCs than the positive and negative controls.

### Example 4

### In-Vitro MSC Stimulation by ECM Protein Extract Vs. Controls Study 2

Bone marrow MSCs were seeded at 6000 cells/cm² in tissue culture dishes with the following culture medium iterations:
(a) 10 μg/ml of bone marrow stromal cell derived ECM protein extract suspended in culture medium
(b) 20 μg/ml of bone marrow stromal cell derived ECM protein extract suspended in culture medium
(c) a negative control with culture medium alone (designated as 2D)
(d) a positive control with seeding on a bone marrow stromal cell derived ECM grown on and attached to the culture dish substrate (designated as 1013.2 HPME) with culture medium.
   Note: The bone marrow stromal cells used to produce the ECM protein extract and the ECM attached to the substrate in Study 2 were from the same donor; and the ECM protein extract was made from the same lot of ECM attached to the substrate.

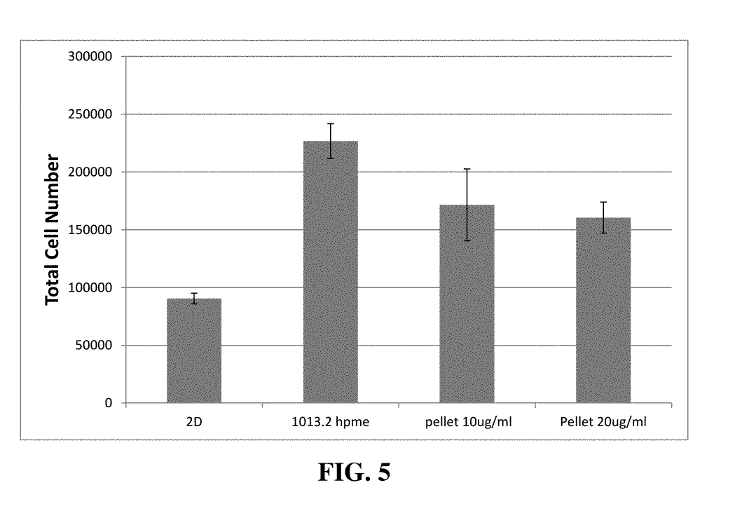

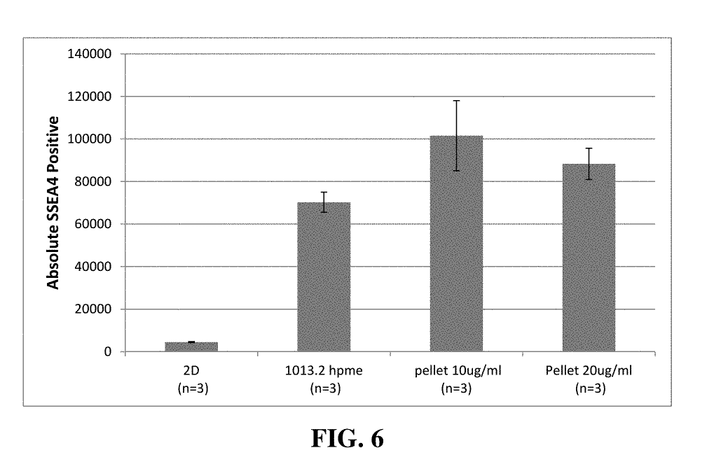

The dishes were incubated at 37° C. for 96 hours after which the cells were detached and counted. The cells were analyzed for SSEA4 expression (MSC marker) using flow cytometry. The data presented are total cell number in FIG. 5 and absolute SSEA4 positive cell number in FIG. 6.

As can be seen from FIG. 6, the bone marrow stromal cell derived ECM protein extract showed greater stimulation of MSCs than the positive and negative controls.

### Example 5

### In-Vivo Orthopedic Study

The bone marrow stromal cell ECM protein extract was evaluated in-vivo in a rat femoral segmental bone defect (SBD) model. The study included three therapy types: Group A—Control, no graft; Group B—Hydroxyapatite/tricalcium phosphate (Medtronic MASTERGRAFT® Mini Granules) (HA/TCP) plus autologous bone marrow; and Group C—HA/TCP plus autologous bone marrow plus bone marrow stromal cell ECM protein extract. Each therapy was implanted in a 6 mm SBD created in the femoral mid-diaphysis of skeletally mature Sprague-Dawley rats (>300 gm). The defect site was stabilized by internal fixation using a pre-drilled polydactyl plate and Kirschner wires, prior to the defect site being sutured closed. The defect sites were wrapped with a collagen membrane (Oestogenics). X-ray images were taken of the defect sites of each study rat periodically.

Post-euthanasia, the femurs were extracted and the femoral mid-diaphysis was scanned by micro computed tomography (micro-CT) in a Skyscan 1176 micro-CT scanner at 9 μm isotropic resolution. Volumetric bone mineral density and trabecular bone volume fraction was measured in a volume of interest that encompasses the 6 mm SBD created. The femurs were also evaluated histologically for extent of mineralization within the defect space.

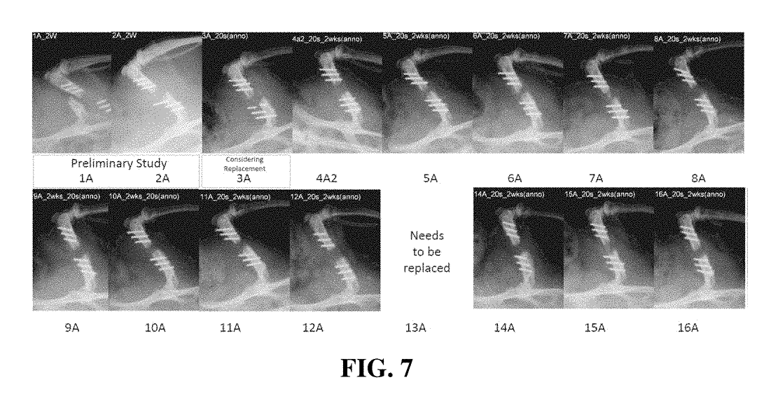

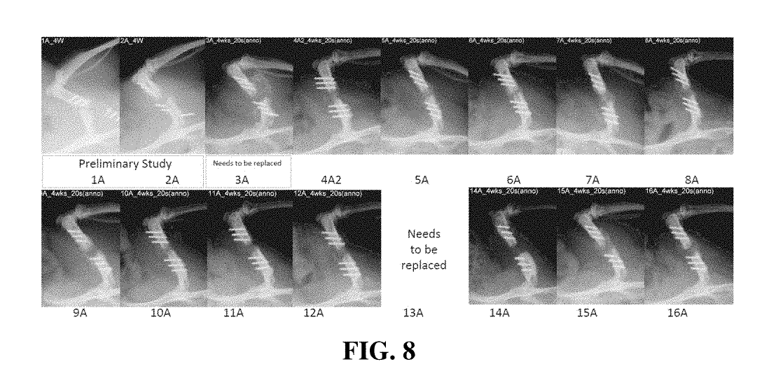

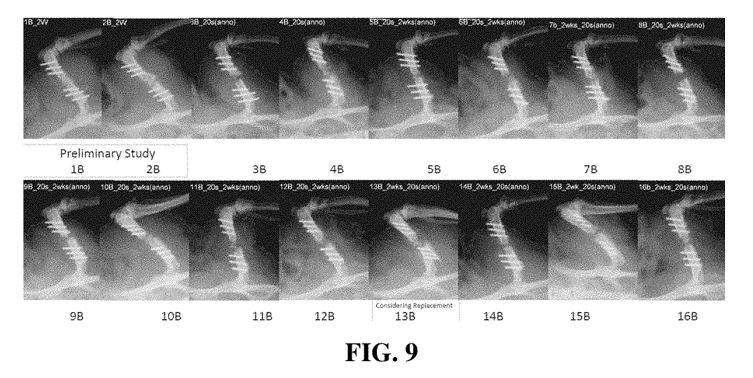

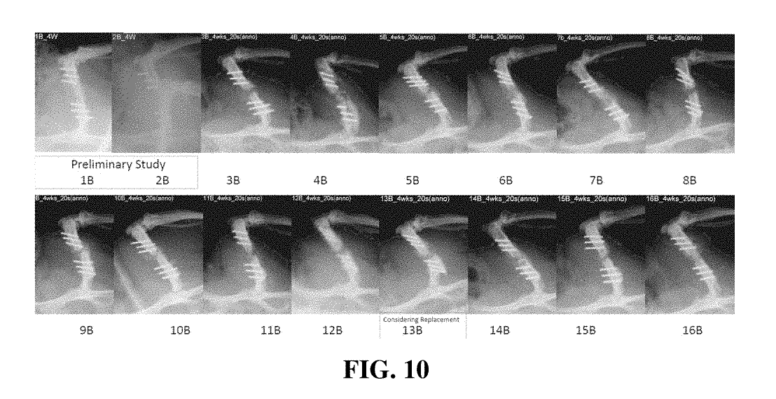

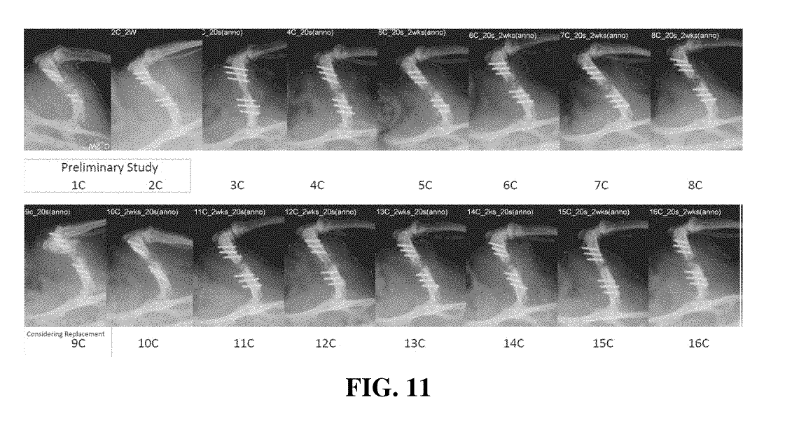

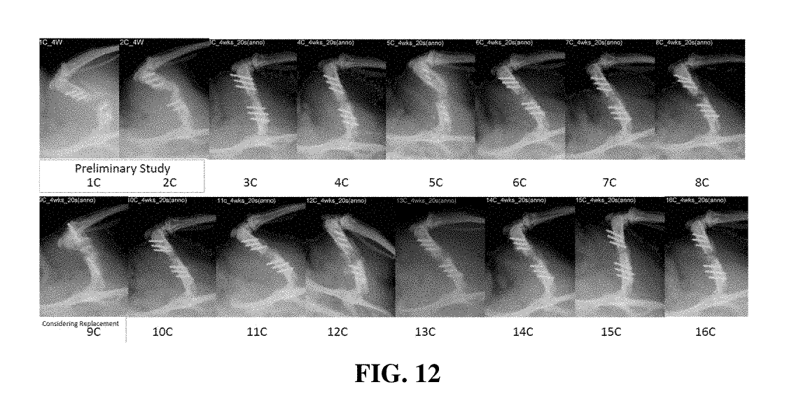

The x-ray images at 2-weeks for Group A (control) are shown in FIG. 7. The x-ray images at 4-weeks for Group A are shown in FIG. 8. The x-ray images at 2-weeks for Group B (HA/TCP) are shown in FIG. 9. The x-ray images at 4-weeks for Group B are shown in FIG. 10. The x-ray images at 2-weeks for Group C (HA/TCP+ECM protein extract) are shown in FIG. 11. The x-ray images at 4-weeks for Group C are shown in FIG. 12.

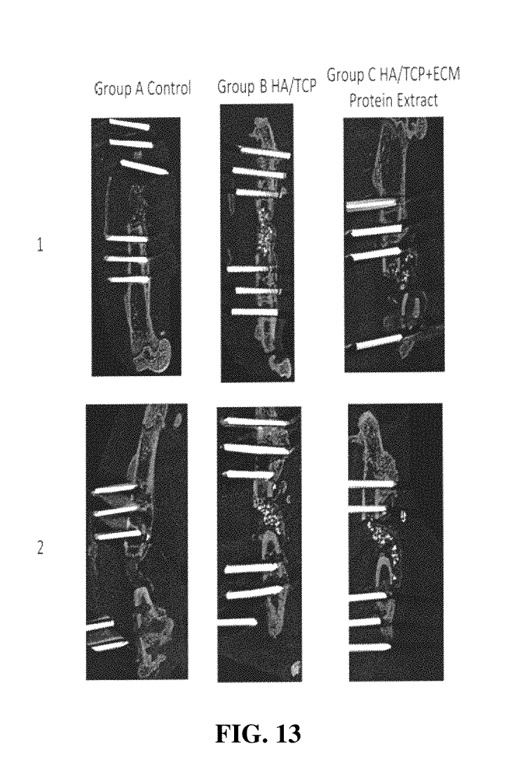

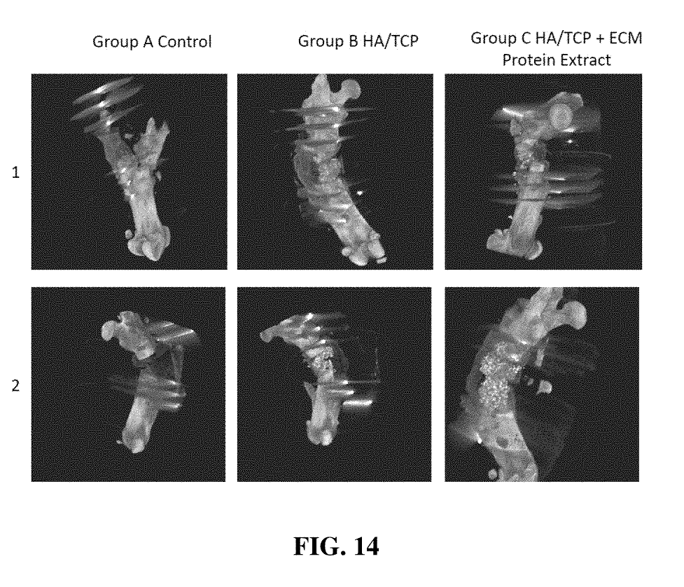

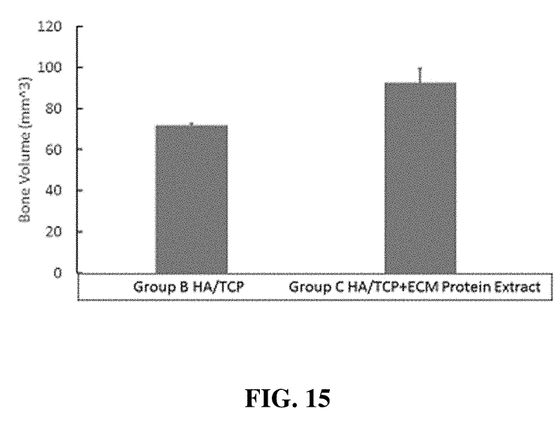

Micro-CT images of Group A (control), Group B (HA/TCP) and Group C (HA/TCP+ECM protein extract) for the first 6 animals of the study after 4 weeks are shown in FIG. 13. Micro-CT composite images of Group A (control), Group B (HA/TCP) and Group C (HA/TCP+ECM protein extract) for the first 6 animals of the study after 4 weeks are shown in FIG. 14. The bone volume in ROI for the first 6 animals of the study after 4 weeks is shown in FIG. 15. As can be seen in FIG. 15, Group C—HA/TCP plus the bone marrow stromal cell derived ECM protein extract showed greater bone regeneration than Group B—with HA/TCP alone.

1.-7. (canceled)

8. A method of making an extracellular matrix (ECM) protein extract, the method comprising:
(a) obtaining viable bone marrow stromal cells;
(b) culturing the bone marrow stromal cells on a substrate to produce a 3D ECM on the substrate;
(c) decellularizing the ECM;
(d) physically removing the ECM from the substrate;
(e) contacting the ECM with an aqueous component with agitation to dissolve and disassociate soluble proteins from the ECM; and
(f) removing the aqueous component from the remaining insoluble portion of the ECM to make the ECM protein extract.

9. The method of claim 8, wherein the substrate is a cell culture container, a plastic cover slip, or microcarriers.

10. The method of claim 8, wherein the substrate is pre-coated with fibronectin.

11.-18. (canceled)

19. The method of claim 8, wherein the ECM protein extract comprises a three-dimensional protein matrix.

20. The method of claim 8, wherein physically removing the ECM from the substrate comprises scraping the ECM off of the substrate.

21. The method of claim 8, wherein removing the aqueous component from the remaining insoluble portion of the ECM comprises pelleting the remaining insoluble portion by centrifugation.

22. The method of claim 8, wherein the agitation of step (e) comprises sonication.

23. A method of expanding mesenchymal stem cells (MSCs) comprising:
(a) obtaining an ECM protein extract according to the method of claim 8; and
(b) contacting the MSCs with the ECM protein extract.

24. A method of generating bone in a subject comprising:
(a) obtaining an ECM protein extract according to the method of claim 8;
(b) combining the ECM protein extract with autologous bone marrow from the subject and a carrier to create a bone-forming composition;
(c) administering the bone-forming composition to the subject.

25. The method of claim 24, wherein the carrier comprises a gel, aqueous liquid, or ceramic powder.

26. The method of claim 24, wherein the carrier comprises hydroxyapatite or hydroxyapatite/tricalcium phosphate.
